(19) United States
(12) Patent Application Publication
(10) Pub. No.: US 2013/0269686 A1
(43) Pub. Date: Oct. 17, 2013

(54) NEBULIZER ASSEMBLIES

(71) Applicant: Thomas Jefferson University Hospitals, Inc., Philadelphia, PA (US)

(72) Inventors: Tiffany Pezzano, Runnemede, NJ (US); Gary Gradwell, Haddonfield, NJ (US); David F. Crosby, Sharon, MA (US)

(21) Appl. No.: 13/750,667

(22) Filed: Jan. 25, 2013

Related U.S. Application Data
(60) Provisional application No. 61/590,611, filed on Jan. 25, 2012.

(51) Int. Cl. A61M 11/02

(52) U.S. Cl. CPC A61M 11/02
USPC 128/200.21

(57) ABSTRACT

Various aspects described herein are directed to nebulizer assemblies for improving the effectiveness of the nebulizers. In some embodiments, instead of placing a nebulizer directly in-line with a first passageway connecting between a patient connector and a ventilator, nebulizer assemblies, in which the nebulizer is connected to a second passageway that extends upward from a top portion of the first passageway, are provided herein. Such configuration of the nebulizer assemblies can prevent a patient's secretion from clogging the nebulizer and thus improve the output of a drug delivered to the patient.

### CROSS-REFERENCE TO RELATED APPLICATIONS

This application claims benefit under 35 U.S.C. §119(e) of provisional application No. 61/590,611, filed on Jan. 25, 2012, the content of which is incorporated herein by reference in its entirety.

### TECHNICAL FIELD OF DISCLOSURE

Various aspects described herein generally relate to nebulizer assemblies used for delivering at least one medication to a patient or a subject in need thereof. More specifically, nebulizer assemblies that are less prone to clogging and/or contamination, e.g., caused by patient's secretions, are described herein.

### BACKGROUND

Patients with respiratory insufficiency often require continuous mechanical ventilation with a ventilator, e.g., a positive-pressure ventilator. A tracheotomy or an endotracheal breathing tube is positioned in the patient's main airway with an internal end positioned for exchange of gas within the lungs and an externally protruding end for connection with a ventilator circuit and then a ventilator system. The ventilator system provides heated, humidified, filtered, breathable gas at a prescribed respiratory rate, tidal volume or pressure, and FiO2 (fraction of inspired oxygen) to the patient in repetitive respiration cycles. In each cycle, gas is delivered through the breathing tube into the lungs of the patients, and then exhaled gas is permitted to passively flow out of the lungs.

Traditionally, jet nebulizers are placed directly in-line with the ventilator circuit between the wye and endotracheal or tracheotomy tube, to deliver a prescribed amount of liquid medication (e.g., bronchodilators or pulmonary vasodilators) into the air stream forced through the inspiratory hose of the ventilator circuit. In such configuration, the nebulizer can become an immobile drain for unwanted materials such as humidity or secretions/blood from a patient, which can clog the nebulizer over time and thus reduce or prevent drug delivery to the patient.

Inhaled nitric oxide (INO) is widely used for critically ill adult patients with severe hypoxemia and/or pulmonary hypertension. Due to the increasing cost of INO, low-cost Flolan (epoprostenol) is more desirable. However, Flolan is a liquid drug that must be nebulized and entrained into the ventilator circuit. Using conventional nebulizer assemblies to administer Flolan to a patient requires close monitoring, because the nebulizer can be clogged with the patient's secretion over time. A clogged nebulizer can prevent the patient from continuously receiving Flolan and result in the patient suffering from poor oxygenation. That is why INO is still a preferred inhaled gas medication for critically ill patients with respiratory illness, which significantly increases the healthcare cost. As such, there is still a need to improve the performance of nebulizer assemblies, e.g., to develop a clog-free nebulizer assembly.

### SUMMARY

Conventional nebulizers placed directly in-line with the ventilators are susceptible to clogging and/or contamination caused by condensation and/or a patient's secretion, e.g., blood, mucus, and saliva, resulting in limited or ineffective drug delivery to a patient. Various embodiments described herein are directed to nebulizer assemblies configured to reduce or inhibit patient secretions or condensation from clogging or draining into a nebulizer, thus improving the function of the nebulizer.

One aspect provided herein relates to a nebulizer assembly comprising (a) a tubular housing having a chamber defining a first passageway in fluid communication with a second passageway, wherein the first passageway connects a first opening of the tubular housing to a second opening of the tubular housing, the first opening being adapted to connect to a patient connector and the second opening being adapted to connect to a ventilator; and wherein the second passageway extends upward from a top portion of the first passageway and connects to a nebulizer port; and (b) a nebulizer having a reservoir for containing a liquid and an outlet adapted to connect to the nebulizer port.

In some embodiments, the first opening of the tubular housing can be located at a first end of the tubular housing. In some embodiments, the second opening of the tubular housing can be located at a second end of the tubular housing. In some embodiments, the second end of the tubular housing can be opposite to the first end of the tubular housing.

In certain embodiments, the first passageway of the tubular housing can further include a drainage port adapted to connect to a secretion container for collecting secretion from a patient. In such embodiments, the drainage port can be located at anywhere on a bottom portion of the first passageway. In one embodiment, the drainage port can be located at anywhere on the bottom portion of the first passageway between the first opening and where the first and the second passageways intersect.

In certain embodiments, the second passageway of the chamber can comprise at least one or more elbows. The elbow(s) can generally form an angle of between about 45 degrees and about 135 degrees. In some embodiments, the elbow(s) can also form an angle smaller than 45 degrees or greater than 135 degrees. In one embodiment, the elbow(s) can form an angle of about 90 degrees.

In some embodiments, the outlet of the nebulizer can be adapted to connect to the nebulizer port of the second passageway such that the nebulizer is positioned vertically or tilted with an angle of less than 90 degrees, e.g., less than 45 degrees.

In some embodiments, the nebulizer assembly can further include at least one or more valves disposed within the chamber. For example, a valve can be disposed anywhere in the first or the second passageway. Such valve can be configured to control a fluid flow within the chamber.

Another aspect provided herein is a nebulizer assembly comprising: (a) a first tubular housing having a first chamber extending between a first opening of the first tubular housing and a second opening of the first tubular housing, a top surface of the first chamber further including a connection port between the first opening and the second opening, wherein the first opening is adapted to connect to a patient connector and the second opening is adapted to connect to a ventilator; (b) a second tubular housing having a second chamber extending between a chamber port of the second tubular housing and a nebulizer port of the second tubular housing, wherein the chamber port is adapted to connect to the connection port of the first chamber; and (c) a nebulizer having a reservoir for containing a liquid and an outlet adapted to the nebulizer port of the second tubular housing.

In some embodiments, the first opening of the first tubular housing can be located at a first end of the first tubular housing. In some embodiments, the second opening of the first tubular housing can be located at a second end of the first tubular housing. In particular embodiments, the second end of the first tubular housing can be opposite to the first end of the first tubular housing.

Optionally, the first chamber of the first tubular housing can further include a drainage port adapted to connect to a container for collecting secretion from the patient. In such embodiments, the drainage port can be configured to be anywhere on a bottom surface of the first chamber. In some embodiments, the drainage port can be configured to be anywhere on a bottom surface of the first chamber between the first opening and the connection port. In one embodiment, the drainage port can be configured to be on a bottom surface opposite to the connection port of the first chamber.

In some embodiments, the second chamber of the second tubular housing can comprise at least one or more elbows between the chamber port and the nebulizer port. The elbow(s) can generally form an angle of between about 45 degrees and about 135 degrees. In some embodiments, the elbow(s) can also form an angle smaller than 45 degrees or greater than 135 degrees. In one embodiment, the elbow(s) can form an angle of about 90 degrees.

In various embodiments, the outlet of the nebulizer can be adapted to connect to the nebulizer port of the second tubular housing such that the nebulizer is positioned vertically, or tilted with an angle of less than 90 degrees, e.g., less than 45 degrees.

In some embodiments, the nebulizer assembly can further include at least one valve disposed within the first and/or the second tubular housing. For example, at least one valve can be configured to be in proximity to a connection between the first and the second chambers. Such valve can be configured to control a fluid flow between the first and the second chambers.

A further aspect described herein relates to nebulizer assemblies without the upward extension of the second passageway from the top portion of the first passageway. In such embodiments, the nebulizer assemblies can comprise (i) a tubular housing having a chamber extending between a first opening of the tubular housing and a second opening of the tubular housing, a bottom surface of the chamber further including a nebulizer port between the first opening and the second opening, and a drainage port between the first opening and the nebulizer port, wherein the first opening is adapted to be connected to a patient connector and the second opening is adapted to connect to a ventilator; (ii) a nebulizer having a reservoir for containing a liquid and an outlet adapted to connect to the nebulizer port; and (iii) a secretion container having a collection chamber for collecting secretion from a patient and an opening adapted to connect to the drainage port.

In some embodiments of any aspects described herein, the liquid contained in the nebulizer reservoir can include at least one drug, e.g., a pulmonary hypertensive drug or a bronchodilator.

The nebulizer assemblies described herein can be generally adapted for use with or without a ventilator. Additionally, the nebulizer assemblies described herein can be adapted for use in any environment, including, but not limited to, at home, in clinics, in hospitals, or during transport from one place to another.

### BRIEF DESCRIPTION OF THE DRAWINGS

Non-limiting embodiments of the nebulizer assemblies will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the inventions. In the figures:

FIG. 1 is a diagrammatic view of a prior-art nebulizer assembly.

FIG. 2A is a diagrammatic view of one embodiment of the nebulizer assembly described herein.

FIG. 2B is a diagrammatic view of the cross-sectional cut indicated in the nebulizer assembly of FIG. 2A.

FIG. 2C is an alternative diagrammatic view of the cross-sectional cut indicated in the nebulizer assembly FIG. 2A.

FIG. 3A is a diagrammatic view of a first tubular housing of the nebulizer assembly in accordance with one embodiment described herein.

FIG. 3B is a diagrammatic view of a second tubular housing of the nebulizer assembly in accordance with one embodiment described herein.

FIG. 3C is an exemplary nebulizer assembly formed by engaging together the first and the second tubular housings shown in FIGS. 3A and 3B, respectively.

FIG. 4 is a diagrammatic view of an alternative embodiment of the nebulizer assembly described herein.

FIG. 5A is a diagrammatic top-view of the nebulizer assembly presented in FIG. 4.

FIG. 5B is a diagrammatic top-view of the nebulizer assembly presented in FIG. 4 having a drainage port of a different cross-sectional shape.

FIG. 6A is a diagrammatic top-view of the nebulizer assembly presented in FIG. 4 having a countersink or a counterbore surrounding the drainage port.

FIG. 6B is a diagrammatic side-view of the nebulizer assembly presented in FIG. 6A.

FIG. 7A is a diagrammatic top-view of the nebulizer assembly presented in FIG. 4 having an incline and a backstop designed around the drainage port.

FIG. 7B is a diagrammatic side-view of the nebulizer assembly presented in FIG. 7A.

### DETAILED DESCRIPTION OF THE INVENTION

Conventional nebulizers can be easily clogged or contaminated by condensation and/or secretions from a patient, e.g., due to a gravitational pull into the nebulizers. Improvement of the existing nebulizer assemblies is thus essential to prevent incidences of nebulizer clogging or contamination. Various embodiments described herein relate to nebulizer assemblies or systems developed to reduce or inhibit the likelihood of a patient's secretion (e.g., saliva, mucus and/or condensation) clogging or draining into a nebulizer, and thus improve the nebulizer output, e.g., increased drug volume delivery to a patient. In some embodiments, the nebulizer assembly is designed such that patients' secretion cannot drain into a nebulizer by gravity, while the flow of drug is maintained between the nebulizer and a patient.

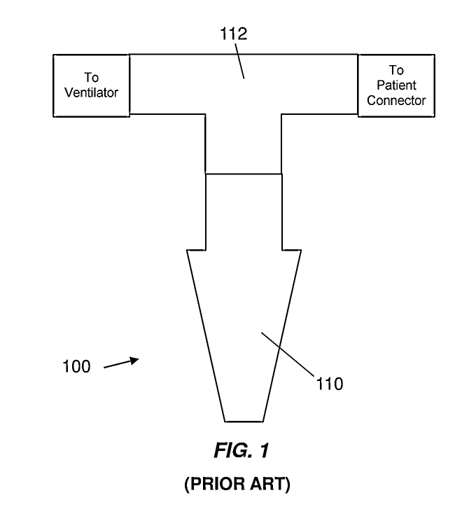

FIG. 1 shows a diagrammatic view of a traditional nebulizer assembly 100, in which a nebulizer 110 is placed directly in-line with a passageway 112 connecting between a patient connector and a ventilator. In such configuration, when a patient respires to receive a medication from the nebulizer 110, a patient's secretion or any condensation produced during respiration can be delivered to and accumulated in the nebulizer assembly 100 over time. The patient's secretion or condensation can be easily drained into the nebulizer 110, e.g., due to a gravitational pull, which will in turn contaminate the medication inside the nebulizer and/or eventually clog the nebulizer to prevent the patient from receiving the medication.

In one aspect, provided herein relates to a nebulizer assembly comprising (1) a tubular housing having a chamber, which includes a first passageway in fluid communication with a second passageway, wherein the second passageway extends upward from a top portion of the first passageway and connects to a nebulizer port; and (2) a nebulizer having a reservoir for containing a liquid and an outlet adapted to connect to the nebulizer port.

For the illustrative purposes only and by no means to be construed as limiting in scope of various aspects and embodiments described herein, references will be made to some embodiments illustrated in the drawing. The inventions described herein include any alterations and further modifications in the illustrated nebulizer assemblies and further applications of the principles associated with various aspects described herein which would normally occur to one skilled in the art.

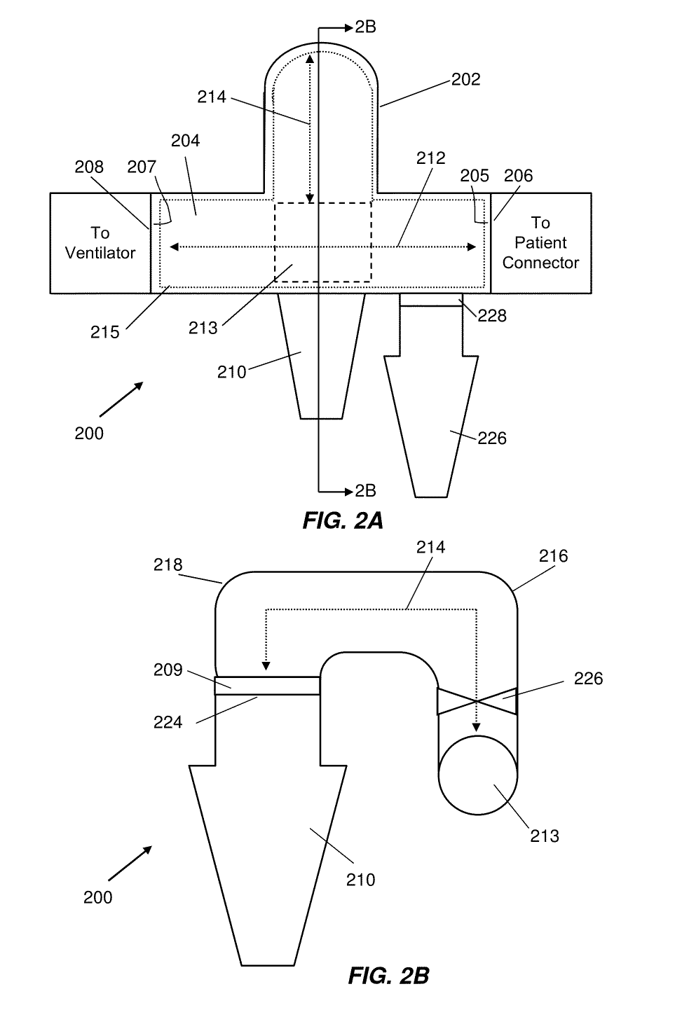

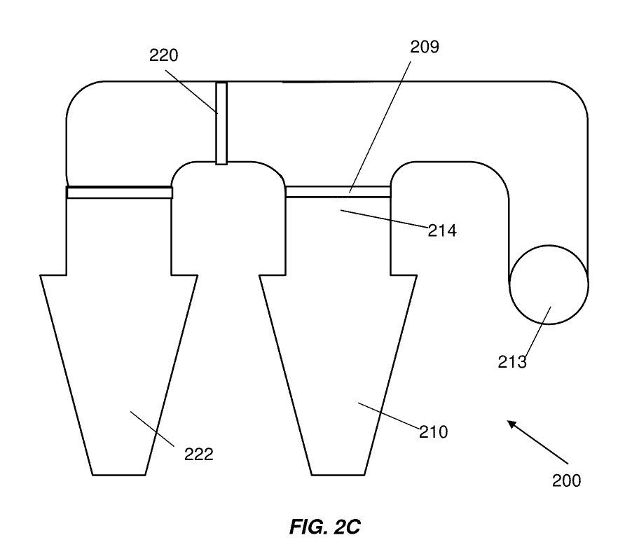

FIGS. 2A-2B show different perspectives of the nebulizer assembly 200 in accordance with one or more embodiments described herein. The nebulizer assembly 200 can include a tubular housing 202, a nebulizer 210, and optionally a secretion container 226. The tubular housing 202 forms a chamber 204 defining a passageway for breathable air and nebulized medication to flow and/or mix together. The nebulizer 210 is a device that is used to convert a liquid medication contained therein into a mist or vapor, which can then be delivered to a patient during respiration. The secretion container 226 can collect a patient's secretion and/or any condensation produced during respiration.

### Tubular Housing of the Nebulizer Assemblies Described Herein

In some embodiments, the tubular housing 202 can form a chamber 204 comprising a first opening 206, a second opening 208, and a nebulizer port 209 for connecting the nebulizer 210 to the chamber 204. The openings (e.g., 206, 208) or ports (e.g., 209) can be generally adapted to connect to various art-recognized devices or equipments to facilitate the delivery of a nebulized medication to a patient. For example, as shown in FIG. 2A, the first opening 206 of the tubular housing can be connected to a patient connector, while the second opening 208 can be connected to a ventilator, if necessary. In some embodiments, the first opening 206 can be located at a first end 205 of the tubular housing (FIG. 2A). In some embodiments, the second opening can be located at a second end 207 of the tubular housing (FIG. 2A). In some embodiments, the second end 207 can be an opposite end of the first end 205 (FIG. 2A).

The first opening 206 of the tubular housing 202 can be adapted to connect to a patient connector. As used herein, the term “patient connector” refers to any device or article that can be used as an interface between a patient and the first opening 206 of the tubular housing. By way of example only, the first opening 206 can be connected to a patient via a tubing (e.g., a flexible tubing) adapted to fit a mouthpiece or a face mask, from which a patient can receive a medication. In some embodiments, at least one or more adapters (e.g., 1 adapter, 2 adapters, 3 adapters or more) can be used to connect the patient connector to the first opening 206 of the tubular housing. As used herein, the term “adapter” refers to any connector for joining at least two parts or devices having different sizes or designs, and enabling them to be fitted or to work together.

The second opening 208 of the tubular housing 202 can be optionally adapted to connect to any respiratory-supporting system that can facilitate the delivery of a nebulized medication to a patient. In some embodiments where a patient has a breathing problem and/or cannot inhale a sufficient amount of a nebulized medication on their own, the second opening 208 can be adapted to connect to a respiratory-supporting system, e.g., a ventilator, to mechanically deliver breathable air and a nebulized medication to the patient. In such embodiments, the second opening 208 can be adapted to connect, e.g., via adapters such as a wye and/or a tubing, to a respiratory-supporting system. The term “wye” is used herein to describe any junction having a single inlet and two outlets as well as any junction having two inlets and a single outlet. In some embodiments, a wye can be a T-shaped junction connector having an angle of 90 degrees between the inlet and both outlet ends or vice versa. Other exemplary respiratory-supporting systems include, but are limited to, the ones described in U.S. Pat. App. Nos.: US 2007/0265611; US 2008/0029096; and US 2010/0269834; and U.S. Pat. No. 5,433,195, the contents of which are incorporated herein by reference.

In other embodiments where a patient can efficiently inhale a nebulized medication on their own, the second opening 208 of the tubular housing 202 needs not be used. In such embodiments, the second opening 208 can be sealed, e.g., with a cap or a stud. In some embodiments, the flow of a fluid (e.g., breathable air and/or nebulized medication) toward the second opening 208 can be regulated or impeded, e.g., by adjusting a controllable valve disposed within the chamber 204 between the second opening 208 and the intersection 213 between the first and the second passageways. The controllable valve can be adjusted, e.g., anywhere between the OPEN and CLOSE position, to control the flow rate of the fluid toward the second opening 208. In some embodiments, the second opening of the tubular housing 208 needs not be included.

The tubular housing 202 can be made or molded from any art-recognized plastic. In some embodiments, the plastics can be transparent, semi-transparent or opaque. Generally, the plastics used to make the tubular housing 202 is desired to be inert (e.g., no reaction to a drug to be delivered or biological secretions from patients). Examples of the plastics can include, but are not limited to, polystyrene, polyethylene, polypropylene, polyethermie, polysulfone, any plastics that are commonly used to manufacture a medical device housing, or a combination thereof. Those skilled in the art will recognize plastic materials that are suitable for the tubular housing 202 described herein.

The cross-sectional shape and/or dimension of the tubular housing 202 can remain constant or vary along the length of the tubular housing 202. In some embodiments, the cross-sectional dimension of the tubular housing 202 (e.g., diameter of the tubular housing) can range from about 10 mm to about 30 mm, from about 12 mm to about 25 mm, or from about 15 mm to about 22 mm. In some embodiments, the cross-sectional dimension of the tubular housing can be larger than 30 mm or smaller than 10 mm. The size or dimension of the tubular housing can be adjusted by a skilled artisan based on a desired flow rate and/or fluid pressure. Without wishing to be bound by theory, a larger tubular housing can be used to achieve or accommodate a larger flow rate and/or a smaller fluid pressure than a smaller tubular housing. For example, a larger tubular housing, e.g., with a dimension between about 25 mm to about 50 mm, can be used to treat large animals such as elephants. In contrast, when treating small animals or children including infants, a smaller tubular housing, e.g., with a dimension between about 8 mm and about 15 mm, can be used if necessary.

The phrase “tubular housing” as used herein refers to a housing with at least one cross section having a tubular shape. As used herein, the term “tubular” is intended to include shapes, including, but not limited to, cylinders. Other shapes that can accomplish the purpose set forth herein, e.g., to allow a fluid flow, can be used. By way of example only, square, rectangular or polygonal-shaped housing is also within the scope of various aspects described herein, and it can be construed as a “tubular” housing as the term is used herein.

### Chamber

The chamber 204 enclosed in the tubular housing 202 provides a pathway for breathable air and nebulized medication to flow through and/or mix together, e.g., between a patient connector and a nebulizer. As shown in FIGS. 2A-2B, the chamber 204 enclosed in the tubular housing 202 defines a first passageway 212 in fluid communication with a second passageway 214. As used herein, the term “fluid communication” between two components (e.g., the first passageway 212 and the second passageway 214) means that a fluid (e.g., gas or liquid) can flow from one component (e.g., the first passageway 212) to another (e.g., the second passageway 214) but does not exclude an intermediate component between the two recited components which are in fluid communication. The term “passageway” as used herein generally refers to a channel, a conduit, a duct, or a pathway through and along which a fluid (e.g., gas or liquid) can flow, pass or move.

In general, the cross section of the chamber 204 that defines a passageway for breathable air and nebulized medication can be formed in any geometrical shape or configuration, for example, tubular (e.g., cylindrical, elliptical), rectangular, triangular, polygonal or a combination thereof. In some embodiments, the cross section of a chamber 204 can generally follow the cross-sectional shape of a tubular housing 202. In some embodiments, the chamber can have a cross-sectional shape different from that of the tubular housing. The dimension or volumetric capacity of the chamber typically depends on the dimension of the tubular housing and/or the thickness of the plastic materials used for the tubular housing. Accordingly, in some embodiments, the cross-sectional dimension of the chamber 204 can range from about 5 mm to about 30 mm, from about 7 mm to about 25 mm, or from about 10 mm to about 22 mm. In some embodiments, the cross-sectional dimension of the chamber can be larger than 30 mm or smaller than 5 mm. The size or dimension of the chamber can be adjusted by a skilled artisan based on a desired flow rate and/or fluid pressure. For example, a larger chamber can be used to treat large animals such as elephants. In contrast, when treating small animals or children including infants, a smaller chamber can be used if necessary.

### Chamber: First Passageway

In embodiments of some aspects described herein, the first passageway 212 within the chamber 204 is in fluid communication with the second passageway 214. Thus, the first passageway 212 can, at least partly, provide a conduit for a nebulized medication delivered from the second passageway 214 to a patient, e.g., via a patient connector. Since the nebulizer 210 is disposed in the second passageway 214, rather than in-line with the first passageway 212 between a patient connector and an optional ventilator as configured in a prior-art nebulizer assembly 100 (FIG. 1), the first passageway 212 within the chamber 204 can also, at least partly, allow a patient's secretion (e.g., saliva, mucus, and condensation) produced during respiration to be collected along the respective chamber wall 215 and/or in a secretion container 226, thus reducing or preventing the patient's secretion from draining into the nebulizer 210 disposed in the second passageway 214. Additionally or alternatively, the first passageway 212 can provide a conduit for breathable air to flow between a patient connector and a ventilator, if necessary.

Accordingly, while the first passageway 212 is in fluid communication with the second passageway 214, in some embodiments, the first passageway 212 can connect a first opening 206 of the tubular housing to a second opening 208 of the tubular housing, wherein the first opening 206 can be adapted to connect to a patient connector. In some embodiments, the second opening of the tubular housing 208 can be optionally connected to a ventilator, if necessary.

In some embodiments, the first passageway 212 can further include a drainage port 228 adapted to connect to the secretion container 226 for collecting secretions (e.g., saliva, mucus, condensation) from a patient. As shown in FIG. 2A, the nebulizer assembly 200 can comprise a secretion container 226 adapted to connect to a drainage port 228. The drainage port 228 can be located anywhere along the first passageway 212, for example, between the first opening 206 and the second opening 208 of the tubular housing as described herein. For example, the drainage port 228 can be located between the first opening of the tubular housing 206 and the intersection 213 of the first passageway and second passageways. Generally, the drainage port 228 can be located at the bottom portion of the first passageway 212, so that any secretion from a patient or condensation can be readily drawn into the secretion container 226 by gravity. In some embodiments, an external pressure, e.g., vacuum, can be further connected to the secretion container 226 to facilitate the collection of secretion from the patient or condensation.

In some embodiments, the secretion containers 226 can be detachable (e.g., for ease of disposal, or for collection of test samples for analysis) and engageable to a drainage port 228 by any means known in the art, e.g., a friction fit, screw-type threads, or any other suitable means known to a skilled artisan. In some embodiments, the drainage port 228 can be adapted to connected to the secretion container 226, e.g., via a tubing and/or an adapter.

The secretion container 226 can be of any shape, e.g., but not limited to, a conical shape, a cylindrical shape, a spherical shape, a rectangular shape, or a polygonal shape. In some embodiments, the secretion container 226 can be a bag. The secretion container 226 can be made of any art-recognized materials, e.g., plastics, glass or any transparent, inert, and durable materials. The secretion container 226 can be of any size, depending on the volume of secretion to be collected over a period of time and/or administration duration of a nebulized medication to a patient. In some embodiments, the secretion container 226 can hold a volume of 5 mL to about 100 mL, or from about 10 mL to about 50 mL.

### Chamber: Second Passageway

In embodiments of some aspects described herein, the second passageway 214 defined by the chamber 204 of the tubular housing extends upward from a top portion of the first passageway 212 and connects to a nebulizer port 209, wherein the second passageway 214 is in fluid communication with the first passageway 212. The upward extension of the second passageway 214 from the top portion of the first passageway 212 can be configured to prevent a patient's secretion or condensation from draining into the nebulizer 210 due to a gravitational pull. As shown in FIGS. 2A-2B, the second passageway 214 can extend upward at any angle from a top portion of the first passageway 212. In some embodiments, the second passageway 214 can extend upward with an angle between 45 degrees and 135 degrees from a top portion of the first passageway 212. In some embodiments, the second passageway can also extend upward from a top portion of the first passageway 212 with an angle less than 45 degrees or greater than 135 degrees (but less than 180 degrees) if necessary. In one embodiment, the second passageway 214 can extend vertically upward (i.e. with an angle of about 90 degrees) from a top portion of the first passageway 212, as shown in FIGS. 2A-2B.

As the second passageway 214 is in fluid communication with the nebulizer 210, the second passageway 214 can additionally act as an outflow reservoir, e.g., for temporary storage or accumulation of a nebulized medication prior to delivery to a patient. Thus, in some embodiments, the addition of the second passageway 214 in the nebulizer assemblies can include an element to control the flow rate of nebulized medication delivered to a patient (e.g., with a valve 226 disposed in the second passageway 214). In some embodiments, the outflow reservoir can allow an increased flow rate of a nebulized medication delivered to a patient.

The second passageway 214 can be of any length or any tortuosity, and it defines the outflow reservoir volume. Without wishing to be bound by theory, the second passageway 214 can be designed to comprise a curved pathway, e.g., in order to maintain the compactness of the nebulizer assemblies while increasing the outflow reservoir. By way of example only, the second passageway 214 can comprise at least one or more elbows, including, for example, one elbow, two elbows, three elbows, four elbows, or more. In some embodiments, the second passageway 214 can comprise at least two elbows 216, 218, for example, as shown in FIG. 2B. The term “elbow” as used herein refers to a structural feature with at least one side of a surface having a curved bend or turn through an angle between 0 degrees and 180 degrees, including, for example, between 45 degrees and about 135 degrees. In some embodiments, the elbow(s) can form an angle of between about 45 degrees and about 135 degrees. In some embodiments, the elbow(s) can form an angle of about 90 degrees. The elbows 216, 218 can be located anywhere along the second passageway 214.

In some embodiments, increasing the fluid volume defined by the second passageway 214, e.g., by increasing the number of elbows (e.g., elbows 216, 218), can increase flow rate of a medication delivered to a patient. However, in other embodiments, too many elbows and/or too short the distance between any two elbows (e.g., elbows 216, 218) could adversely reduce the flow rate of a medication delivered to a patient because of the tortuosity of the resultant second passageway. The optimum number and locations of the elbows and/or the optimum distance between any two elbows formed within the second passageway can be determined by a skilled artisan to achieve an optimum drug delivery rate, e.g., using mathematical modeling, based on a number of factors, including, but not limited to, desirable fluid dynamics, fluid flow direction and/or fluid volume defined by the second passageway 214.

In various embodiments, the second passageway 214 can further comprise an additional opening or port (e.g., port 220). The additional opening or port can be adapted for various applications or purposes. By way of example only, as shown in FIG. 2C, the additional opening or port 220 can be adapted for connecting to a second nebulizer 222. In such embodiments, the second nebulizer 222 can contain a different liquid medication, or the same liquid medication, e.g., to increase the total drug volume delivered to a patient. In other embodiments, the additional opening or port 220 can be adapted for use as a vent or drainage. In alternative embodiments, the additional opening or port 220 can be sealed or closed with a detachable cap, when it is not used.

As shown in FIGS. 2B-2C, the nebulizer port 209 of the second passageway 214 and the outlet of the nebulizer 224 can be engaged to each other by any suitable means known in the art. For example, they can be engaged to each other by screw-type threads, adhesive, friction fit or any other suitable means known to a skilled artisan. In some embodiments, the outlet of the nebulizer 214 can be engaged to the nebulizer port 209 via an adapter to accommodate the difference in their opening sizes. In some embodiments, the nebulizer port 209 of the second passageway can be adapted to connect to the outlet 224 of the nebulizer such that the nebulizer 210 is positioned vertically. In some embodiments, the nebulizer port 209 of the second passageway can be adapted to connect to the outlet 224 of the nebulizer such that the nebulizer 210 is positioned with a tilting angle of less than 90 degrees.

### Nebulizer

Any art-recognized nebulizer can be used in the nebulizer assemblies described herein. The nebulizer 210, 222, 310 can include a receptacle or a reservoir for containing one or more liquid medications, which are indicated to be inhaled rather than ingested, e.g., a pulmonary hypertensive drug, corticosteroids and bronchodilators. Additionally, the nebulizer 210, 222, 310 can include a mechanical unit to convert a liquid medication into an aerosol or a mist, which can then be inhaled by a patient. For example, a jet nebulizer can include a compressor, which causes compressed air or oxygen to flow at high velocity through a liquid medication and thus turn the liquid medication into an aerosol or a mist.

In some embodiments, the nebulizer 210, 222, 310 can include an air intake tube for receiving air from an external source and delivering it to an atomizer within the nebulizer for discharging atomized or nebulized medication through the outlet of the nebulizer.

In some embodiments, the nebulizers 210, 222, 310 can include at least one port for addition of desired materials and/or removal of liquids from the nebulizer reservoir. For example, the nebulizer 210, 222, 310 can include a medication port, which allows additional liquid medication to be added to the nebulizer while the nebulizer is engaged to the rest of the nebulizer assembly. The medication port can further include a flexible piece (e.g., rubber) on an outer end of the port such that it can be punctured, e.g., with a syringe or similar articles, to deliver medication through the port. After delivery of medication through the port, the punctured flexible piece can be resealable, e.g., to prevent contamination of the medication. In some embodiments, the nebulizer 210, 222, 310 can also include a suction port for removing residual medication from the reservoir after a particular dose of medication has been dispensed from the nebulizer reservoir. Such suction port can further include a suction tube having a first end accessible to the bottom of the reservoir.

In some embodiments, the nebulizer 210, 222, 310 can be detached from the nebulizer assembly as described herein, e.g., detachable from the tubular housing 202. Thus, the nebulizer 210, 222, 310 can be easily disconnected from the rest of the nebulizer assembly, e.g., for disposal, cleaning, and/or filling with a medication. Alternatively, the nebulizer 210, 222, 310 and the tubular housing 202 can be formed together as a single piece.

### Alternative Embodiments

In alternative embodiments, the tubular housing 202 having a chamber 204 that defines both the first 212 and the second 214 passageways as described herein can be formed in any ways known in the art. For example, the tubular housing 202 can be formed as an integral unit (e.g., a single piece as shown in FIGS. 2A-2B). Alternatively, the tubular housing 202 can be formed by assembling together a plurality of subunits, e.g. at least 2 subunits, at least 3 subunits, at least 4 subunits. By way of example only, the tubular housing 202 having a chamber 204 that defines the first 212 and the second 214 passageways can be formed by providing at least two subunits such as two individual tubular housings 302A and 302B, each of which, can, respectively, define a first and second passageway 312, 314 and can be adapted to connect to each other. Accordingly, in another aspect, provided herein is a nebulizer assembly comprising (a) a first tubular housing 302A having a first chamber 304A extending between a first opening 306 of the first tubular housing 302A and a second opening 308 of the first tubular housing 302A, a top surface of the first chamber 304A further including a connection port 313A between the first opening 306 and the second opening 308, wherein the first opening 306 is adapted to connect to a patient connector and the second opening 308 is adapted to connect to a ventilator; (b) a second tubular housing 302B having a second chamber 304B extending between a chamber port 313B of the second tubular housing 302B and a nebulizer port 309 of the second tubular housing 302B, wherein the chamber port 313B is adapted to connect to the connection port 313A of the first chamber 304A; and (c) a nebulizer 310 having a reservoir for containing a liquid and an outlet 324 adapted to the nebulizer port 309 of the second tubular housing 302B.

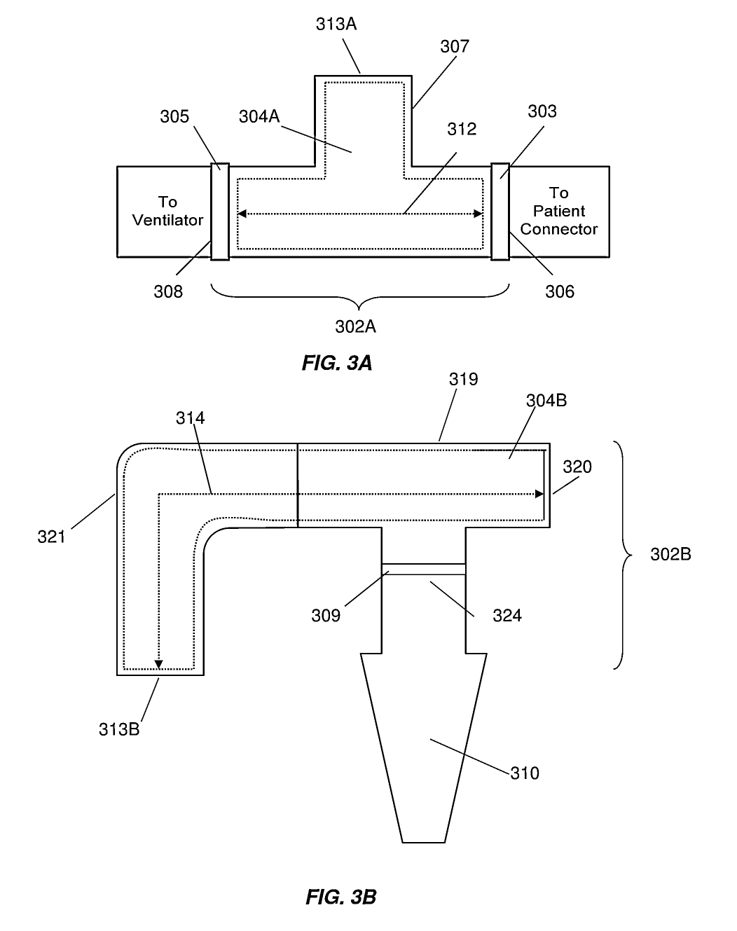

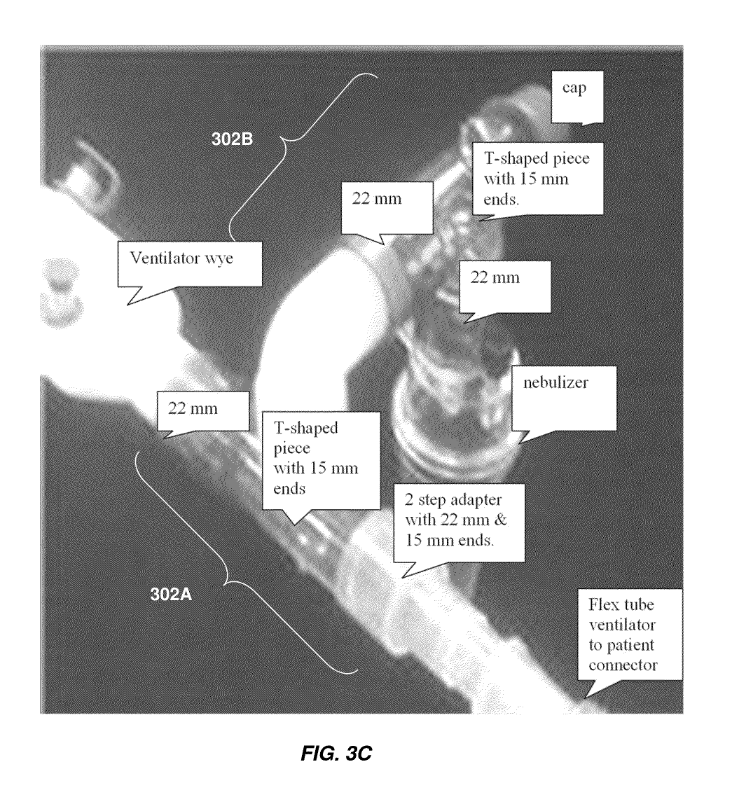

The first and/or the second tubular housing 302A, 302B can each be an integral unit, or can each be formed or assembled together from a plurality of connecting pieces, including bent or T-shaped connectors 307, 319 and/or adaptors 303, 305. In some embodiments, as shown in FIG. 3A, the first tubular housing 302A can be formed by assembling together at least two adaptors 303 and 305 and at least a first inverted T-shaped connector 307 comprising a connection port 313A. The first opening 306 of the first tubular housing 302A can be adaptably connected to a patient connector, whereas the second opening 308 of the first tubular housing 302A can be adaptably connected (e.g., via a ventilator wye) to a ventilator. Different size of adaptors can be used, alone or in combination, in the nebulizer assemblies described herein. In some embodiments, the adaptors (e.g., adaptors 303, 305) can have a size ranging from about 10 mm to about 30 mm. In some embodiments, a smaller adaptor (e.g., with a size of about 12 mm to about 18 mm) can be fitted into a larger adaptor (e.g., with a size of about 20 mm to about 25 mm) in order to accommodate different opening sizes of the two connecting components on both ends.

In one embodiment, as shown in FIG. 3B, the second tubular housing 302B can be formed by assembling together at least one bent connector 321 comprising a chamber port 313B and another end adaptably connected to a second T-shaped connector 319 comprising a nebulizer port 309.

In operation, an exemplary nebulizer assembly, for example, as shown in FIG. 3C, can be formed by engaging the chamber port 313B of the assembled second tubular housing 302B to the connection port 313A of the assembled first tubular housing 302A such that a passageway is created for a fluid (e.g., nebulized medication) to flow between the first tubular housing 302A and the second tubular housing 302B. The nebulizer port 309 can then be adaptably connected to a nebulizer 310 as described herein. The spare port 320 of the second T-shaped connector 319 can be capped, or be adaptably connected to a second nebulizer or drainage. A liquid medication contained in the nebulizer 310 will then be nebulized, exit through the outlet of the nebulizer 324 and continue to flow through the chamber 304B of the second tubular housing 302B toward the chamber 304A and the first opening 306 of the first tubular housing 302A that is adaptably connected to a patient connector (e.g., a mouthpiece or face mask). For a patient who has difficulties with breathing, the second opening 308 of the first tubular housing 302A can be further adaptably connected to a ventilator. Otherwise, the second opening 308 of the first tubular housing 302A can be capped or closed as described earlier.

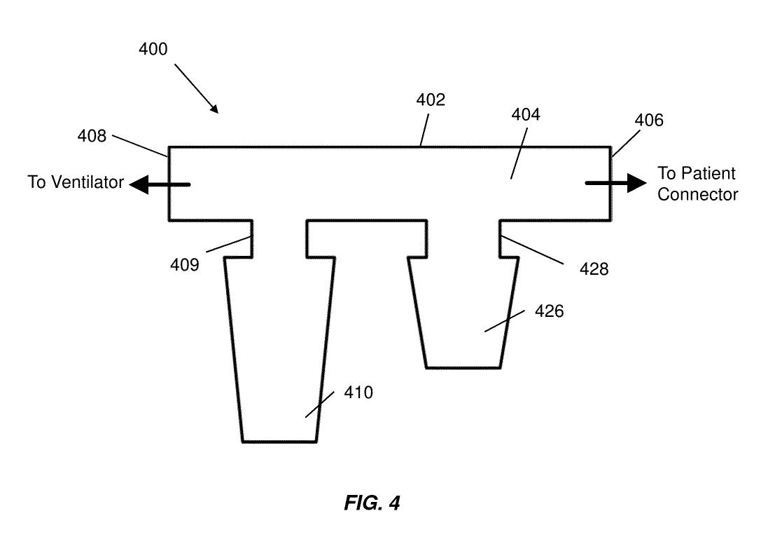

In an alternative embodiment as shown in FIG. 4, a nebulizer assembly 400 can comprise (i) a tubular housing 402 having a chamber 404 extending between a first opening 406 of the tubular housing and a second opening 408 of the tubular housing, a bottom surface of the chamber 406 further including a nebulizer port 409 between the first opening and the second opening, and a drainage port 428 between the first opening and the nebulizer port, wherein the first opening is adapted to connect to a patient connector and the second opening is adapted to connect to a ventilator; (ii) a nebulizer 410 having a reservoir for containing a liquid and an outlet adapted to connect to the nebulizer port 409; and (iii) a secretion container 426 having a collection chamber for collecting secretion from a patient and an opening adapted to connect to the drainage port 428.

In some embodiments, the nebulizer assembly 400 can comprise at least one drainage port 428, e.g., at least two, at least three or more drainage ports, between the first opening 406 of the tubular housing and the nebulizer port 409.

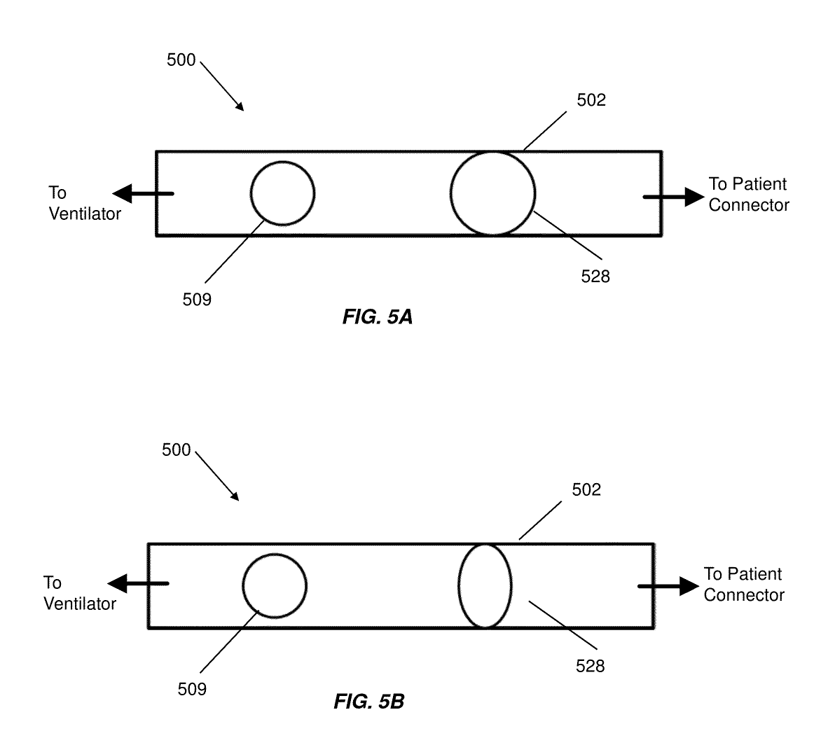

The drainage port can have a cross-section of any shape, e.g., a circle, an ellipse, a triangle, a square, a rectangle, a polygon or any irregular shape. In some embodiments, the drainage port 528 can have a circular cross-section, as shown in FIG. 5A. In other embodiments, the drainage port 528 can have an elliptical or oval cross-section, as shown in FIG. 5B. One of skill in the art can determine an optimum cross-sectional shape of the drainage port, based on a number of design parameters, e.g., the opening shape of the secretion container and/or adaptors, and fluid mechanics parameters such as fluid flow rate.

The cross-sectional dimension of the drainage port can be of any length. By way of example only, when the drainage port has a circular cross-section, the cross-sectional dimension (e.g., diameter) of the drainage port 528 can be substantially identical to the cross-sectional dimension (e.g., diameter) of the tubular housing 502. The terms “substantially identical” is used in reference to a cross-section of the drainage port 528 having a dimension (e.g., diameter) sufficient to capture the desired volume of secretion. In one embodiment, the drainage port diameter can be at least about 80% of the cross-sectional dimension of the tubular housing 502, including at least about 85%, at least about 90%, at least about 95%, at least about 98%, at least about 99% or more, or any integer between 80% and 100%, of the cross-sectional dimension of the tubular housing 502. In some embodiments, the term “substantially identical” can include 100%.

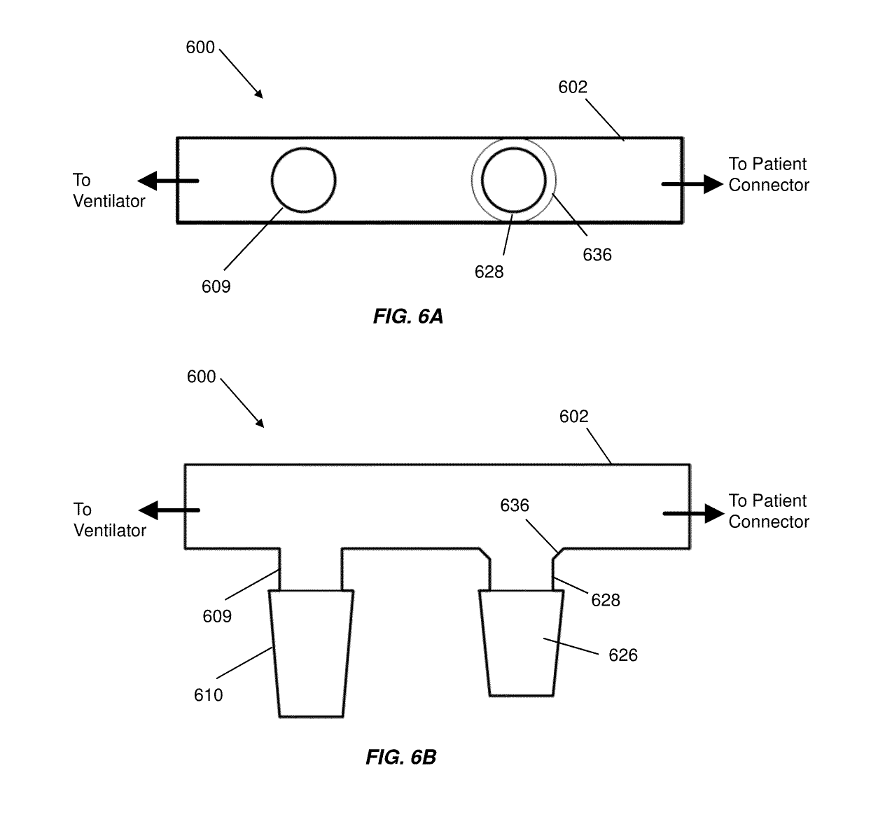

A person having ordinary skill in the art would appreciate that, based on the principles of fluid mechanics, a fluid (e.g., secretion) is more likely to flow toward the drainage port if the cross-sectional dimension of the drainage port is substantially identical or same as the cross-sectional dimension of the drainage port. However, in other embodiments where the cross-sectional dimension of the drainage port 628 is smaller than the cross-sectional dimension of the tubular housing 602, e.g., the cross-sectional dimension of the drainage is at least about 10% smaller than the cross-sectional dimension of the tubular housing (as shown in FIGS. 6A-6B), at least one countersink 636, or one or more counterbores, can be used to enlarge the opening of the drainage port 628, to transition over the difference in the cross-sectional dimension between the drainage port 628 and the tubular housing 602.

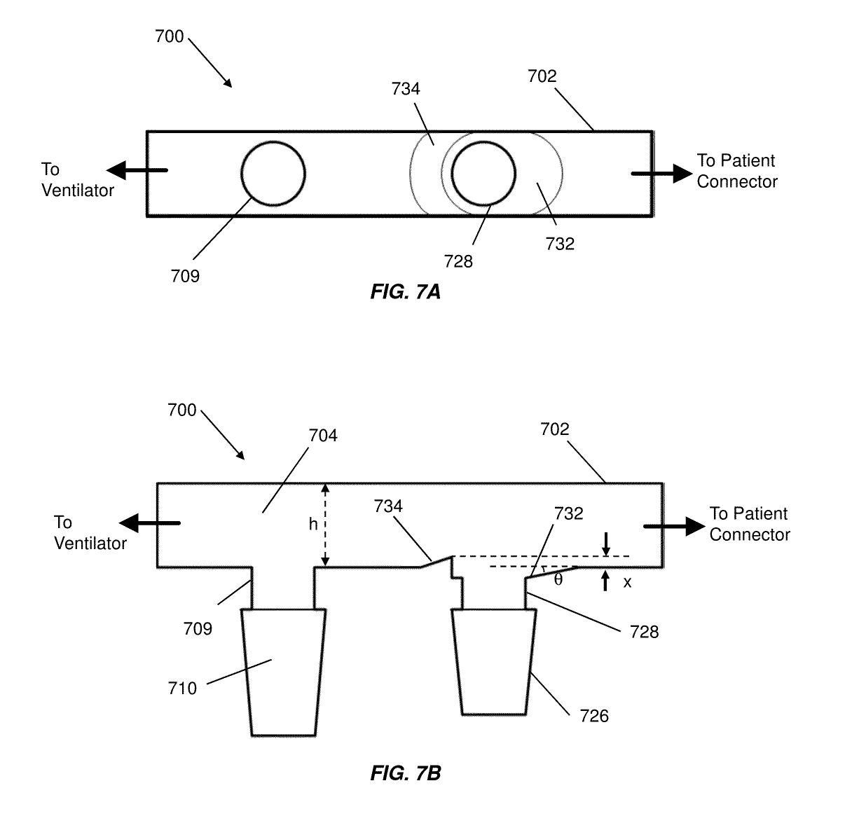

In some embodiments, as shown in FIGS. 7A and 7B, to facilitate a fluid (e.g., secretion) flowing toward the drainage port 728 of any size, an incline 732 can be added to direct flow into the drainage port 728 along the bottom surface of the chamber 704. In some embodiments, the incline can, for example, have an angle θ between 5 degrees and 30 degrees, relative to the bottom surface of chamber 704, although angles greater than 30 degrees or less than 5 degrees can be used. One of skill in the art can optimize the angle θ of the incline based on fluid mechanics calculation including a number of factors, such as fluid viscosity and flow rate, length of the incline, dimension of the drainage port, relative position of the drainage port along the tubular housing, and any combinations thereof.

In some embodiments, to minimize or prevent the likelihood of a fluid (e.g., secretion) escaping from the drainage port and flowing toward the nebulizer, the bottom surface of the chamber 704, as shown in FIGS. 7A and 7B, can further comprise at least one backstop 734, including at least two, at least three or more backstops, between the nebulizer port 709 and the drainage port 728. Preferably, the height x of the backstop 734, relative to the bottom surface of the chamber 704, can be within 5%-15% of the height h of the tubular housing. However, in some embodiments, according to each particular design, the height x of the backstop 734, relative to the bottom surface of the chamber 704, can be smaller than 5% of the height h of the tubular housing or larger than 15% of the height h of the tubular housing, with the upper limit being a function of the desired air flow requirements of the ventilator system. As used herein, the term “backstop” refers to any structural element that can serve as a stop or barrier to minimize a fluid (e.g., secretion) from flowing back into the nebulizer.

In some embodiments of any aspects described herein, the nebulizer assembly can further comprise at least one valve disposed within the chamber (e.g., chamber 204, 304A, and/or 304B). The term “valve,” as used herein, includes any passive or actuated fluid flow controller or other actuated mechanism for selectively passing a fluid through an opening, including, without limitation, ball valves, plug valves, butterfly valves, choke valves, check valves, gate valves, leaf valves, piston valves, poppet valves, rotary valves, slide valves, solenoid valves, 2-way valves, or 3-way valves. Valves can be actuated by any method, including, without limitation, by mechanical, electrical, magnetic, camshaft-driven, hydraulic, or pneumatic means.

The valve can be disposed anywhere within the chamber (e.g., chamber 204, 304A, and/or 304B) according to how a skilled artisan desires to control a fluid flow. By way of example only, as shown in FIG. 2B, if a skilled artisan desires to control the flow rate of a nebulized medication from the nebulizer port 209, via the second passageway 214, to the first passageway 213 connected to a patient connector, a valve 226 can be disposed within the second passageway 214. Additionally, a valve can be disposed within the first passageway 212 if it is desirable to control the flow rate therein.

The nebulizer assemblies described herein can be generally adapted for use in any subject in need thereof, e.g., mammals such as human subjects. In some embodiments, the size of the nebulizer assemblies described herein can be adapted for use by any subject. For example, the size of the nebulizer assemblies, e.g., the cross-section of the tubular housing, can be made smaller for small children or infants in order to reduce the amount of delivered medication. Additionally, the nebulizer assemblies described herein can be adapted for use in any environment. In some embodiments, the nebulizer assemblies described herein can be adapted for portable use. In some embodiments, the nebulizer assemblies described herein can be adapted for use in a hospital setting. In some embodiments, the nebulizer assemblies described herein can be adapted for home use.

Embodiments of the various aspects described herein can be illustrated by the following numbered paragraphs.

1. A nebulizer assembly comprising:
a. a tubular housing having a chamber defining a first passageway in fluid communication with a second passageway, wherein
   the first passageway connects a first opening of the tubular housing to second opening of the tubular housing, the first opening being adapted to connect to a patient connector and the second opening being adapted to connect to a ventilator; and wherein
   the second passageway extends upward from a top portion of the first passageway and connects to a nebulizer port; and
b. a nebulizer having a reservoir for containing a liquid and an outlet adapted to connect to the nebulizer port.

2. The nebulizer assembly of paragraph 1, wherein the second passageway comprises at least one elbow.

3. The nebulizer assembly of paragraph 1 or 2, wherein the elbow forms an angle of between about 45 degrees and about 135 degrees.

4. The nebulizer assembly of any of paragraphs 1-3, wherein the elbow forms an angle of about 90 degrees.

5. The nebulizer assembly of any of paragraphs 1-4, wherein the outlet of the nebulizer is adapted to connect to the nebulizer port such that the nebulizer is positioned vertically.

6. The nebulizer assembly of any of paragraphs 1-5, wherein the first opening is located at a first end of the tubular housing.

7. The nebulizer assembly of any of paragraphs 1-6, wherein the second opening is located at a second end of the tubular housing.

8. The nebulizer assembly of paragraph 7, wherein the second end is an opposite end of the first end.

9. The nebulizer assembly of any of paragraphs 1-8, further comprising a secretion container for collecting secretion from the patient.

10. The nebulizer assembly of paragraph 9, wherein the secretion container is adapted to connect to a drainage port located at a bottom portion of the first passageway.

11. The nebulizer assembly of any of paragraphs 1-10, further comprising a valve disposed within the chamber.

12. The nebulizer assembly of paragraph 11, wherein the valve is configured to control a fluid flow in the chamber.

13. The nebulizer assembly of paragraph 11 or 12, wherein the valve is disposed in the first or the second passageway.

14. The nebulizer assembly of any of paragraphs 1-13, wherein the liquid contains at least one drug.

15. The nebulizer assembly of paragraph 14, wherein the drug is a pulmonary hypertensive drug.

16. A nebulizer assembly comprising:
a. a first tubular housing having a first chamber extending between a first opening of the first tubular housing and a second opening of the first tubular housing, a top surface of the first chamber further including a connection port between the first opening and the second opening, wherein the first opening is adapted to connect to a patient connector and the second opening is adapted to connect to a ventilator;
b. a second tubular housing having a second chamber extending between a chamber port of the second tubular housing and a nebulizer port of the second tubular housing, wherein the chamber port is adapted to connect to the connection port of the first chamber; and
c. a nebulizer having a reservoir for containing a liquid and an outlet adapted to the nebulizer port of the second tubular housing.

17. The nebulizer assembly of paragraph 16, wherein the second chamber of the second tubular housing comprises at least one elbow between the chamber port and the nebulizer port.

18. The nebulizer assembly of paragraph 16 or 17, wherein the elbow forms an angle of between about 45 degrees and about 135 degrees.

19. The nebulizer assembly of any of paragraphs 16-18, wherein the elbow forms an angle of about 90 degrees.

20. The nebulizer assembly of any of paragraphs 16-19, wherein the outlet of the nebulizer is adapted to connect to the nebulizer port of the second tubular housing such that the nebulizer is positioned vertically.

21. The nebulizer assembly of any of paragraphs 16-20, wherein the first opening is located at a first end of the first tubular housing.

22. The nebulizer assembly of any of paragraphs 16-21, wherein the second opening is located at a second end of the first tubular housing.

23. The nebulizer assembly of paragraph 22, wherein the second end is an opposite end of the first end.

24. The nebulizer assembly of any of paragraphs 16-23, wherein the first chamber of the first tubular housing further includes a drainage port adapted to connect to a secretion container for collecting secretion from the patient.

25. The nebulizer assembly of paragraph 24, wherein the drainage port of the first chamber is located on a bottom surface of the first chamber.

26. The nebulizer assembly of any of paragraphs 16-25, further comprising a valve disposed within the first chamber.

27. The nebulizer assembly of any of paragraphs 16-26, further comprising a valve disposed within the second chamber.

28. The nebulizer assembly of any of paragraphs 26-27, wherein the valve is configured to control a fluid flow between the first and the second chambers.

29. The nebulizer assembly of any of paragraphs 16-28, wherein the liquid contains at least one drug.

30. The nebulizer assembly of paragraph 29, wherein the drug is a pulmonary hypertensive drug.

31. A nebulizer assembly comprising:
a. a tubular housing having a chamber extending between a first opening of the tubular housing and a second opening of the tubular housing, a bottom surface of the chamber further including a nebulizer port between the first opening and the second opening, and a drainage port between the first opening and the nebulizer port, wherein the first opening is adapted to be connected to a patient connector and the second opening is adapted to connect to a ventilator;
b. a nebulizer having a reservoir for containing a liquid and an outlet adapted to connect to the nebulizer port; and
c. a secretion container having a collection chamber for collecting secretion from a patient and an opening adapted to connect to the drainage port.

32. The nebulizer assembly of paragraph 31, wherein a cross-sectional dimension of the drainage port is substantially identical to a cross-sectional dimension of the tubular housing.

33. The nebulizer assembly of paragraph 31, wherein the cross-sectional dimension of the drainage port is smaller than the cross-sectional dimension of the tubular housing.

34. The nebulizer assembly of paragraph 33, wherein the drainage port further comprises a counterbore such that a cross-sectional dimension of the counterbore is substantially identical to the cross-sectional dimension of the tubular housing.

35. The nebulizer assembly of any of paragraphs 31-34, wherein the bottom surface of the chamber further comprises at least one backstop between the nebulizer port and the drainage port.

While there has been shown and described in some embodiments of a nebulizer assembly, it will be appreciated that many changes and modifications can be made therein without, however, departing from the essential spirit thereof. Thus, the inventions are not limited to the particular embodiments disclosed herein, for it can be realized that various size and/or shapes of the tubular housing and/or nebulizer can be used for the purposes of the inventions. The terminology used herein is for the purpose of describing particular embodiments only, and is not intended to limit the scope of the present inventions, which is defined solely by the claims.

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

All numbers expressing quantities used herein should be understood as modified in all instances by the term “about.” The term “about” when used in connection with percentages may mean ±1%.

The following examples are intended to illustrate certain embodiments of the nebulizer assemblies described herein, but do not exemplify the full scope of the inventions described herein.

### EXAMPLE 1

This example demonstrates one or more embodiments of the nebulizer assembly described herein and the efficiency of using the same, as compared to a traditional nebulizer assembly 100, in which the nebulizer is placed in-line with the ventilator circuit.

Traditionally, jet nebulizers for bronchodilators or pulmonary vasodilators are placed directly in-line with the ventilator circuit between the wye and endotracheal/tracheal tube. This often acts as an immobile drain for unwanted materials such as humidity or secretions/blood. This also results in reduced drug output to the patient secondary to the absence of a reservoir and jet clogging.

In one instance, the typical jet nebulizer assembly was clogged by secretion, which in turn prevented a pulmonary hypertensive patient from receiving epoprostenol sodium (or Flolan) resulting in poor oxygenation.

As such, one embodiment of the nebulizer assembly described herein has been developed (FIG. 3C). In such embodiment, a first inverted T-shaped connector piece 307 is connected between a wye of the ventilator and an endotracheal tube to form a first passageway 312 as described herein. The connection port 313A of the first inverted T-shaped connector piece 307 is then connected to a chamber port 313B of a 90-degree swivel or rotatable connector 321, with another end connected to a second T-shaped connector piece 319 to form a second passageway 314 as described herein. The nebulizer port 309 of the second T-shaped connector piece 319 is connected to a nebulizer 310, acting as an outflow reservoir from the nebulizer 310.

Such configuration of the nebulizer assembly can prevent unwanted drainage (clogging) into the nebulizer 310. The second passageway 314 acting as an outflow reservoir can increase drug volume delivered to a patient and thus improve nebulizer output. Further, the placement of the nebulizer is distal to the circuit hearted wire at a lower temperature and humidity level, thus increasing aerosol drug delivery. The clinical observations have shown positive patient outcomes without clogging the nebulizer over an extended period of time.

In the claims, as well as in the specification above, all transitional phrases such as “comprising,” “including,” “having,” and “containing,” are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Various changes and modifications to the disclosed embodiments, which will be apparent to those of skill in the art, may be made without departing from the spirit and scope of the present invention. Further, all patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A nebulizer assembly comprising:
a. a tubular housing having a chamber defining a first passageway in fluid communication with a second passageway, wherein
the first passageway connects a first opening of the tubular housing to second opening of the tubular housing, the first opening being adapted to connect to a patient connector and the second opening being adapted to connect to a ventilator; and wherein
the second passageway extends upward from a top portion of the first passageway and connects to a nebulizer port; and
b. a nebulizer having a reservoir for containing a liquid and an outlet adapted to connect to the nebulizer port.

2. The nebulizer assembly of claim 1, wherein the second passageway comprises at least one elbow.

3. The nebulizer assembly of claim 2, wherein the elbow forms an angle of between about 45 degrees and about 135 degrees.

4. The nebulizer assembly of claim 2, wherein the elbow forms an angle of about 90 degrees.

5. The nebulizer assembly of claim 1, wherein the outlet of the nebulizer is adapted to connect to the nebulizer port such that the nebulizer is positioned vertically.

6. The nebulizer assembly of claim 1, wherein the first opening is located at a first end of the tubular housing.

7. The nebulizer assembly of claim 1, wherein the second opening is located at a second end of the tubular housing.

8. The nebulizer assembly of claim 7, wherein the second end is an opposite end of the first end.

9. The nebulizer assembly of claim 1, further comprising a secretion container for collecting secretion from the patient.

10. The nebulizer assembly of claim 9, wherein the secretion container is adapted to connect to a drainage port located at a bottom portion of the first passageway.

11. The nebulizer assembly of claim 1, further comprising a valve disposed within the chamber.

12. The nebulizer assembly of claim 11, wherein the valve is configured to control a fluid flow in the chamber.

13. The nebulizer assembly of claim 1, wherein the liquid contains at least one drug.

14. The nebulizer assembly of claim 13, wherein said at least one drug comprises a pulmonary hypertensive drug.

15. A nebulizer assembly comprising:
a. a tubular housing having a chamber extending between a first opening of the tubular housing and a second opening of the tubular housing, a bottom surface of the chamber further including a nebulizer port between the first opening and the second opening, and a drainage port between the first opening and the nebulizer port, wherein the first opening is adapted to be connected to a patient connector and the second opening is adapted to connect to a ventilator;
b. a nebulizer having a reservoir for containing a liquid and an outlet adapted to connect to the nebulizer port; and
c. a secretion container having a collection chamber for collecting secretion from a patient and an opening adapted to connect to the drainage port.

16. The nebulizer assembly of claim 15, wherein a cross-sectional dimension of the drainage port is substantially identical to a cross-sectional dimension of the tubular housing.

17. The nebulizer assembly of claim 15, wherein the cross-sectional dimension of the drainage port is smaller than the cross-sectional dimension of the tubular housing.

18. The nebulizer assembly of claim 17, wherein the drainage port further comprises a counterbore such that a cross-sectional dimension of the counterbore is substantially identical to the cross-sectional dimension of the tubular housing.

19. The nebulizer assembly of claim 15, wherein the bottom surface of the chamber further comprises at least one backstop between the nebulizer port and the drainage port.
